(19) United States
(12) Patent Application Publication
(10) Pub. No.: US 2006/0051776 A1
(43) Pub. Date: Mar. 9, 2006

(54) Gene manipulation method using homologous recombination

(75) Inventors: Douglas J. DeMarini, Berwyn, PA (US); Steven A. Sheardown, Harlow (GB)

Correspondence Address: GLAXOSMITHKLINE;Corporate Intellectual Property -UW2220, P.O. Box 1539, King of Prussia, PA, 19406-0939, US

(73) Assignee: SmithKline Beecham Corporation

(21) Appl. No.: 11/063,045

(22) Filed: Feb. 22, 2005

Related U.S. Application Data
(63) Continuation of application No. 10/069,767, filed on Feb. 22, 2002, now abandoned.
(60) Provisional application No. 60/152,851, filed on Sep. 8, 1999.

(51) Int. Cl. C12Q 1/68; C12P 21/06; C12N 15/00

(52) U.S. Cl. 435/6; 435/69.1; 435/440

(57) ABSTRACT

Provided is a method of inserting an insert polynucleotide into a target nucleic acid having a first end and a second end by homologous recombination.

The present invention relates to methods of inserting a polynucleotide segment into a vector using homologous recombination.

To study a given gene or gene product, researchers must often clone precise DNA sequences from one vector to another. The discovery of the polymerase chain reaction (PCR) has allowed researchers to readily clone such sequences. Such cloning is typically done by introducing recognition sequences for restriction endonucleases at the ends of oligonucleotides that anneal to the DNA sequence of interest. These oligonucleotides are used as primers to amplify the sequences of interest. After amplification, the DNA product is usually purified, restriction digested, and ligated into a desired vector. This process has been used extensively by countless labs, and the technique has improved with the development of higher fidelity polymerases, less expensive oligonucleotides, and more reliable, user-friendly thermocyclers.

Although PCR-based cloning has been the workhorse of gene cloning for the last decade, it nevertheless has its drawbacks. For instance, some DNA sequences are simply recalcitrant to amplification by PCR. Because PCR products are ultimately ligated into a restriction-enzyme digested vector, the cloning is dependent upon the availability of restriction sites that are present in the vector, but absent in the sequences to be cloned. Additionally, it is sometimes difficult to obtain relatively large, error-free PCR products. Thus, cloning of some DNA sequences can require several primer pairs, multiple PCR reactions and correction of PCR-induced errors. In some cases, one must resort to time-consuming, traditional, enzyme-based cloning. Moreover, PCR amplification of even short DNA sequences can create a mutated product. Consequently, researchers who require faithful replication of the DNA sequences of interest must ultimately perform DNA sequence analysis for the entire length of each cloned PCR product. Although DNA sequence analysis has become cheaper and faster, it is still time consuming, and it is not economically feasible for many labs to sequence the entirety of every PCR-amplified clone.

To create a method for rapidly cloning many genes into a given vector, and which reduces the amount of DNA sequence analysis for the resulting clones, a method that uses yeast-based homologous recombination has now been developed.

Homologous recombination in cells such as those of the yeast Saccharomyces cerevisiae is an extremely efficient process that has been exploited by geneticists for years, and was recently used to clone nearly all of the know S. cerevisiae open reading frames (ORFs) (Hudson et al., Genome Res. 7, 1169-1173, 1997). This technique, known as gap repair (Orr-Weaver et al., Proc. Natl. Acad. Sci. USA 78, 6354-6358, 1983; FIG. 1A), joins the sequences of interest to a yeast vector by homologous recombination. As such this cloning method joins DNA sequences in a restriction-enzyme independent fashion. Although several variations of gap repair have been developed to clone novel DNA sequences (e.g., Ma et al., Gene 58, 201-216, 1987; Hudson et al., 1997; Marykwas and Passmore, Proc. Natl. Acad. Sci. USA 92, 11701-11705, 1995; Fusco et al., Yeast 15, 715-720, 1999) most require PCR. As such, these methods can be impeded by the size of the DNA sequences to be cloned, the occasional difficulties of “tailed” PCR, and the risk of PCR-induced error.

A recent modification to gap repair (Raymond et. al., BioTechniques 26, 134-141, 1999) describes the use of “recombination linkers” to stimulate homologous recombination between the yeast vector and the DNA sequences of interest (FIG. 1B). This procedure maintains the restriction-enzyme independent feature of gap repair, delimits the size of the DNA sequences to be cloned and requires sequence analysis only for the regions of recombination. Thus, to confirm that the exact sequence has been cloned, only two sequencing reactions need be performed on any contiguous piece of cloned DNA, regardless of its size.

Raymond et al. found that overlaps (i.e., recombination sequences according to the nomenclature used in the present application) of ≧50 bp were particularly effective, though overlaps of 30 bp were somewhat effective.

Nevertheless, synthesis of the recombination linkers requires a relatively large number of oligonucleotides (e.g., 4-8 per cloning), and PCR-mediated amplification, thereby rendering the final product susceptible to PCR-induced errors. Herein is described an improvement on recombination linker-based cloning that allows PCR-independent synthesis of the recombination linkers, and requires fewer oligonucleotides.

### SUMMARY OF THE INVENTION

The invention provides for example, a method of inserting an insert polynucleotide into a target nucleic acid having a first end and a second end by homologous recombination comprising: inserting into a recombination-competent cell the following first nucleic acids: one or more insert polynucleotides each comprising an insert segment, and at least one linker comprising (i) a substantially single-stranded recombination sequence, (ii) a recombination sequence of no more than 25 base pairs, or (iii) a combined length of no more than 45 nucleotides; and generating within the cell a nucleic acid containing the insert segments inserted between the first and second ends in an order defined by recombination sequences found in the target nucleic acid and the first nucleic acids. For example, the insert polynucleotide can be inserted into genomic nucleic acid.

In one embodiment, the invention relates to a method of inserting an insert polynucleotide into a vector by homologous recombination comprising: inserting into a recombination-competent cell the following first nucleic acids: one or more insert polynucleotides each comprising an insert segment, a vector-related nucleic acid having a first end and a second end, at least one linker comprising (i) a substantially single-stranded recombination sequence, (ii) a recombination sequence of no more than 25 base pairs, or (iii) a combined length of no more than 45 nucleotides; and generating within the cell a vector containing the insert segments inserted between the first and second ends in an order defined by recombination sequences found in the first nucleic acids.

In another embodiment, the invention relates to a method of cloning or subcloning an insert nucleic acid comprising: isolating an insert polynucleotide comprising the (i) insert nucleic acid and, (ii) at least at one end of the insert nucleic acid, an additional polynucleotide portion; inserting into a recombination-competent cell the following first nucleic acids: the insert polynucleotide, a vector-related nucleic acid having a first end and a second end, at least one linker comprising (i) a substantially single-stranded recombination sequence, (ii) a recombination sequence of no more than 25 base pairs, or (iii) a combined length of no more than 45 nucleotides; and generating within the cell a vector containing the insert nucleic acid inserted between the first and second ends in an order defined by recombination sequences found in the first nucleic acids.

In still another embodiment, the invention relates to a method of cloning or subcloning an insert nucleic acid: isolating (i) a first insert polynucleotide comprising (a) a first insert segment that defines a portion of the insert nucleic acid and, at one or both ends of the first insert segment, (b) additional polynucleotide sequence, and (ii) one or more additional insert polynucleotides comprising additional insert segments that define portions of the insert nucleic acid, wherein the insert segments can be aligned to generate the insert nucleic acid; inserting into a recombination-competent cell the following first nucleic acids: the first insert polynucleotides and the additional insert polynucleotides, a vector-related nucleic acid having a first end and a second end, at least one linker comprising (i) a substantially single-stranded recombination sequence, (ii) a recombination sequence of no more than 25 base pairs, or (iii) a combined length of no more than 45 nucleotides; and generating within the cell a vector containing the insert nucleic acid inserted between the first and second ends in an order defined by recombination sequences found in the first nucleic acids.

### BRIEF DESCRIPTION OF THE DRAWINGS

FIGS. 1A and 1B display gap repair using (A) “tailed” PCR products and (B) double-stranded linkers. X's indicate regions of cross over stimulated by homologous recombination. Double lines (B) represent PCR-amplified, double-stranded recombination linkers. Overhanging lines (C) represent complementary oligonucleotides. Boxes and straight lines represent DNA sequences of interest.

FIG. 1C illustrates a gap-repair/recombination-mediated method of making an insertion using complementary oligonucleotides as a linker.

FIG. 2 diagrams (A) primer sets used to PCR amplify double-stranded recombination linkers, and (B) primer pairs used as recombination linkers. Dashed lines represent DNA sequences of pRS423, solid lines represent DNA sequences from a NotI fragment of pCDN. Arrow heads designate the 3′ ends. Numbers in parenthesis indicate the length of complementation between two oligonucleotides. Oligonucleotide numbers (see, Example, Table 1) are shown.

FIG. 3 shows the results of inserting various combinations of polynucleotides into a cell appropriate to effect recombination.

FIG. 4 shows an illustration of gene replacement in an animal.

FIG. 5 shows the construction of a targeting vector.

### DEFINITIONS

The following terms shall have, for the purposes of this application, the respective meaning set forth below.
first and second ends of a vector-related nucleic acid. The “ends” of a vector-related nucleic acid are the ends of the segment of such vector-related nucleic acid that is intended to be part of a product of the method described herein.
insert segment. An insert segment is that portion of an insert polynucleotide or a linker that is incorporated with the vector-related nucleic acid. A “P-insert segment” is derived from an insert polynucleotide, while a “L-insert segment” is derived from a linker.

It will be recognized that the recombination sequences, since they comprise identical or highly similar sequences between two polynucleotides, can be found on both the two polynucleotides that contributed to nucleic acid at a given junction formed by the process of the invention. Thus, identifying which “segment” came from which polynucleotide could be difficult; however, such strict accounting is unnecessary to the invention. For ease of visualization, one can designate one strand of the product nucleic acid the reference strand, and, for each segment, all sequence on that reference strand to the 5′ end consistent with the corresponding insert polynucleotide can be designated as part of the segment. As will be evident, the 3′ end of a segment is defined under this rule by the endpoint of the next (5′ to 3′) insert segment.
insert nucleic acid. An insert nucleic acid is that formed as an insert into a vector-related nucleic acid by the processes of the invention. The insert nucleic acid derives from insert segments found in insert polynucleotides or linkers introduced into the recombination-competent cell. The insert segments are joined together and to each other in an order defined by recombination sequences. In one particular embodiment of the invention, there is one insert polynucleotide that yields an insert segment that is the insert nucleic acid.
insert polynucleotide. An insert polynucleotide is one of one or more polynucleotides introduced into the recombination-competent cell that contributes an insert segment to the insert nucleic acid.
linker. A linker is a relatively short nucleic acid, typically chemically synthesized (e.g., by solid phase synthesis) or amplified that is designed to have two recombination sequences that direct the attachment of an P-insert segment to either a target nucleic acid (such as a vector-related nucleic acid) or another P-insert segment via the resulting L-insert segment.
recombination-competent cell. A recombination-competent cell is a cell, such as a cell from at least certain yeast strains, that is competent to repair plasmids based on the complementary overlap.
recombination sequence. A recombination sequence is a sequence found on one of the nucleic acids acted on by an insertion process of the invention {i.e., (i) vector-related nucleic acid, (ii) insert polynucleotides or (iii) linkers}, where this sequence has a length of sufficiently similar sequence to another recombination sequence found on another of these cell-inserted nucleic acids to facilitate recombination to join the two nucleic acids.
   Preferably, such a recombination sequence is at least 20 nucleotides in length, or at least 30, 40, 60 or 150 nucleotides, and is at least 95, 97, 98 or 99% identical (more preferably at least 100% identical) to its cognate recombination sequence.
substantially single-stranded. A substantially single-stranded linker segment is a recombination sequence, where a portion is designed to not complement another linker nucleic acid that is inserted into the cell. Preferably the single-stranded portion consists of at least five (5) nucleotides, more preferably ten (10) nucleotides, yet more preferably twenty (20), thirty (30) or fifty (50) nucleotides. Preferably, the single-stranded portion provides at least a 2-fold enhancement in the efficiency with which the intended circular nucleic acid is achieved.
vector-related nucleic acid. A vector-related nucleic acid has, when the first and second ends of such vector-related nucleic acid are joined by an insert polynucleotide, the elements required for replication or maintenance of a vector within a cell.
aligmment, where one aligned polynucleotide is single-stranded. Sequence alignments with a single-stranded segment can be with the strand that has the single-stranded segment, or with that strand's full complement, including that which would base-pair with the single-stranded segment.
insert segments can be aligned. Insert segments from insert polynucleotides “can be aligned” if sequence overlaps found in the insert polynucleotides or in conjunction with linkers define a given continuous sequence after switching from one nucleic acid to another at the overlaps. The sequence overlaps should be of sufficient sequence similarity and length to serve a recombination sequences. Such a continuous sequence might be that of the desired insert nucleic acid.
relatedness. Relatedness between two or more polypeptide sequences or two or more polynucleotide sequences can be measured in terms of “identity” or, for polypeptides, in terms of “similarity.” In the art, the term “identity,” which refers to a subset of “similarity,” is used in connection with the output of various computer programs that compare sequences and seek to align the sequences. The “identity” determined by such programs can vary with the program parameters which define the value judgments used to make the alignment. With highly related sequences, however, and in the context of defining the present invention, such value judgments are less important. This is because, while the term is used to indicate strong relatedness. This relatedness does not need to meet any criteria for evolutionary relatedness. Also, the calculation can be an absolute calculation of identity between two strings of sequence, to give the largest match between the sequences tested without discarding any reductions for non-matches between the sequences. The calculation can be definitive because at the high levels of identity in question it is practical to make the alignment that achieves the largest match.

By way of example, a polynucleotide sequence of the present invention can be identical to a reference sequence (such as a reference nucleic acid sequence or a relevant segment thereof), that is it can be 100% identical, or it can include up to a certain integer number of nucleotide alterations as compared to the reference sequence such that the percent identity is less than 100% identity. Each such alteration can be the deletion, substitution (including transition or transversion [a purine for a pyrimidine or vice versa]), or insertion of a single nucleotide, and wherein such alterations can occur relative to the 5′ or 3′ termini of the reference polynucleotide sequence or anywhere between the terminal positions, interspersed either individually among the nucleic acids in the reference sequence or in one or more contiguous groups within the reference sequence. As an illustration, by a polynucleotide having a nucleotide sequence having at least, for example, 95% “identity” to a reference nucleotide sequence of SEQ ID NO: 1, it is intended that the nucleotide sequence of the polynucleotide is identical to the reference sequence except that the polynucleotide sequence can include an average of up to five nucleotide alterations (point mutations) per each 100 nucleotides of the reference nucleotide sequence of SEQ ID NO: 1.

Thus, unless another meaning is specified, this application will speak of relatedness between two strings of sequence (a query string and a reference string) in terms of a query sequence that is identical with the reference sequence, or, if not identical, then over the entire length corresponding to the reference sequence, the nucleic acid sequence has an average of up to thirty (or twenty, ten, five, two or one) substitutions, deletions or insertions for every 100 nucleotides or amino acid residues of the reference sequence. In one embodiment there is one substitution, deletion or insertion for every 200 nucleotides or amino acid residues of the reference sequence. This measure can be viewed as 70% identity when there are thirty alterations per hundred, 80% when there are twenty alterations, 90% when there are 10 alterations, 95% when there are 5 alterations, 98% when there are 2 alterations, 99% when there is 1 alteration per hundred, and 99.5% identity when there is 1 alteration per two hundred.

To exemplify, consider the following:

R XXXABCD EFGHIJKLMNOPQRSTYYYY
Q CDZZZEFGHIJKL OPQRSTXXXXXXXXX
Score ----00110001111111100111111-----------

The double-underlined portion of the line labeled “R” represents the reference sequence. The query sequence Q is aligned with the reference sequence so as to get the highest match given the rule that any mismatch against the reference sequence reduces the match score. Thus, the two nonmatches to “AB” and the two non-matches against “MN” reduce scoring by four points, and the three non-matched insertions in the query sequence reduce scoring by three. Thus, since there are twenty residues in the reference sequence, percent identity is:

$$\frac{( 20 - 7 )}{20} \times 100 \% = 65 \%$$

More generally, % identity can be calculated as [1−Nₙ/Xₙ]×100; wherein Nₙ is the number of nucleotides in query polynucleotide sequence that are substituted, deleted or inserted when compared to the reference polynucleotide, and Xₙ is the number of nucleotides in the reference sequence.

Alternatively, “Identity” can be readily calculated by known methods. including but not limited to those described in: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math, 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Watermnan algorithm can also be used to determine identity.

Parameters for polynucleotide comparison include the following:
Algorithm: Needleman and Wunsch, J. Mol Biol. 48: 443-453 (1970)
Comparison matrix: matches=+10, mismatch=0
Gap Penalty: 50
Gap Length Penalty: 3
   Available as: The “gap” program from Genetics Computer Group, Madison Wis. These are the default parameters for nucleic acid comparisons.

### DETAILED DESCRIPTION OF THE INVENTION

The method of the invention uses short or single-stranded linkers with homologous sequence to each of two polynucleotides that one seeks to have linked. The homologous stretches facilitate homologous, vector repairing, recombination. Preferably, there is a selective pressure for creating a desired vector or other product nucleic acid incorporating the desired inserts. Under an embodiment of the method of the invention, at least one such region of homology (or recombination sequence) is substantially single-stranded. This single-stranded feature allows one to eliminate or limit the amount of enzyme-based amplification used to create double stranded linkers. In one preferred embodiment, the linker is made up of a first oligonucleotide that is substantially single-stranded but has a base-pairing overlap with another oligonucleotide that completes the linker. Note, however, that the two oligonucleotides of this embodiment do not have to be annealed prior to insertion into the cell that drives recombination. In one preferred embodiment, the linker is made of two annealing oligonucleotides, one of which is substantially single-stranded, where the base-pairing overlap between the two oligonucleotides has a portion of both recombination sequences.

In another embodiment, one of the recombination sequences of a linker 25 nucleotides or less, preferably 20 nucleotides or 15 nucleotides or less. Under this embodiment, homologous recombination occurs with surprisingly short recombination sequences are effective, thereby rendering it more practical to supply the linker oligonucleotides without use of an amplification reaction that serves to lengthen a double-stranded linker. Note that under this embodiment, the short recombination sequences can be fully double-stranded.

In another embodiment, the total length of the linker, taking into account both strands as associated by overlap, can be 45 nucleotides or less in length, or 40 or 30 nucleotides or less in length. This embodiment again provides polynucleotide linkers that are surprisingly shorter than those taught in the prior art.

Results using the present invention describe a method of DNA cloning that can be PCR-independent and restriction-enzyme independent. In addition, this method of the inventions shares two attractive feature of double-stranded linker-based cloning: DNA sequences can be transferred regardless of their length, GC content, or structure; and sequence confirmation for any construct can be achieved with only two sequencing reactions, regardless of the length of cloned DNA. Indeed, in analyses conducted on products of the inventive method, DNA sequence errors were identified only in the regions of the oligonucleotides, even though sequence information was obtained for at least 200 nucleotides on either side of these sequences. These errors could result from mis-incorporation during oligonucleotide synthesis, or an error in homologous recombination.

Additionally, when compared to the method using double-stranded linkers, the method of the invention can reduce the number of oligonucleotides required, and can eliminate PCR reactions and subsequent analysis. When compared to traditional PCR-based cloning, the method eliminates or minimizes purification, restriction, and ligation of PCR-amplified products. Although amplification-dependent methods are now time-honored and well-known, PCR amplification can introduce errors into the sequences of interest, and the procedures can be time consuming. The procedure described here is relatively non-labor intensive, requiring little more than designing primers, transforming yeast, rescuing plasmids from yeast to E. coli, and analyzing plasmids. Although the procedure can require several days to identify plasmids with the appropriate restriction pattern, much of that time is spent culturing cells. Thus, many different DNA sequences can be handled simultaneously.

A potential drawback is that a vector used in the method should be appropriate for a given recombination-competent cell, such as a yeast-based plasmid. As such, for researchers who ultimately want to express a particular DNA sequence in another organism, the methods of the invention can produce a shuttle vector that contains sequences required for selection, replication, and expression in other organisms. Alternatively, methods of the invention can be used to transfer DNA sequences into a yeast-modified univector (Qinghua et al, Curr. Biol. 8, 1300 1309, 1998), thus facilitating transfer into a multitude of other plasmids.

Routine adjustment of the length of complementarity, length of homology between the oligonucleotides, insert, and vector, or adjustment of selection parameters (such as cloning into a counter-selectable marker (e.g., URA3)), can improve the process by, for example, reducing any background of undesired vectors.

The results obtained with the invention show that non-amplified oligonucleotides stimulate homologous recombination. Given that non-complementary oligonucleotides were unable to stimulate homologous recombination efficiently (if at all), it appears that the complementary oligonucleotides must anneal to each other prior to recombination. It is not known if the oligonucleotides anneal in vivo or ex vivo. Annealing the oligonucleotides prior to transformation has not, at least in some experiments, increased the frequency of recombination. Thus, in these instances, it appears that the oligonucleotides anneal as well in the transformation reaction mixture or inside the cells as they do in an annealing reaction. Without being limited to theory, it is possible that the annealed oligonucleotides are made completely double stranded by DNA polymerases in vivo.

Recent developments such as high-throughput sequencing, sophisticated sequence-analysis programs, microarray technologies, and the broadening interest in model organisms have rapidly increased the already large number of interesting genes. To keep pace, systems such as Seamless cloning (Stratagene), Topo-cloning (Invitrogen), the univector plasmid-fusion system (Qinghua et al, 1998, supra), and homologous recombination-based cloning have been created or improved to facilitate transfer of precise DNA sequences from vector to vector. To date, these systems all depend upon either PCR of the sequences of interest, require that the sequences of interest be flanked by particular DNA sequences, or require that the sequences of interest exist in a particular vector prior to transfer. The cloning system of the present invention alleviates all these requirements, and has multiple applications. For instance, not only can sequences be transferred from vector to vector, but as is true for double-stranded linkers (Raymond et. al.), it is likely that they can be modified by including in the complementary oligonucleotides sequences for restriction sites, ribosome binding, preferred codons, epitope tags, etc. In addition to gene cloning, tagging, and synthesis (Raymond et. al.), this system can also be used to transfer entire gene libraries or pools of cDNA into yeast vectors. Thus, one is able to capture specific, full-length cDNAs using sequence data from both or either ends of a gene, and to insert relatively large DNA sequences into circular yeast plasmids, thereby facilitating synthesis of otherwise labor-intensive constructs, such as those used to knock out mouse genes.

In certain embodiments, the recombination sequences are preferably at least 30 nucleotides in length, more preferably at least 40, 50, 60 or 80 nucleotides in length. See, Raymond et. al., BioTechniques 26, 134, 1999, Fusco et. al., Yeast 15, 715, 1999, for recommended minimum lengths when using double-stranded linkers. When a linker is composed of two polynucleotides having an overlapping complementary region, that overlapping region is preferably at least 10 nucleotides in length, more preferably at least 15, 20, 25, 26, 35 or 50 nucleotides in length.

The linkers are preferably placed in the cellular insertion (e.g., transfection or transformation) reaction in molar excess of the insert polynucleotides, for instance a 1,000-fold, 2,000-fold or 3,000-fold molar excess. The insert polynucleotides are preferably placed in the reaction at a molar excess of the vector-related nucleic acid, for instance a 50, 100 or 200 molar excess. The vector-related nucleic acid can be contacts with the cells in an amount, for example, of 0.1 μg, 0.15 μg or 0.2 μg per 4×10⁹ cells.

The recombination-competent cells can be readily and routinely identified by application of a method of the invention that has been effective in a cell known to be competent. Recombination-competent cells include, for example, those of the following species: S. cerevisiae (including the YEF473 strain), S. pombe, C albicans and E. coli.

Application of the Recombination Method to Construct Targeting Vectors

In one preferred embodiment of the invention, the methods are used to construct targeting vectors for replacing genomic sequence in an animal, such as a mammal (e.g., human).

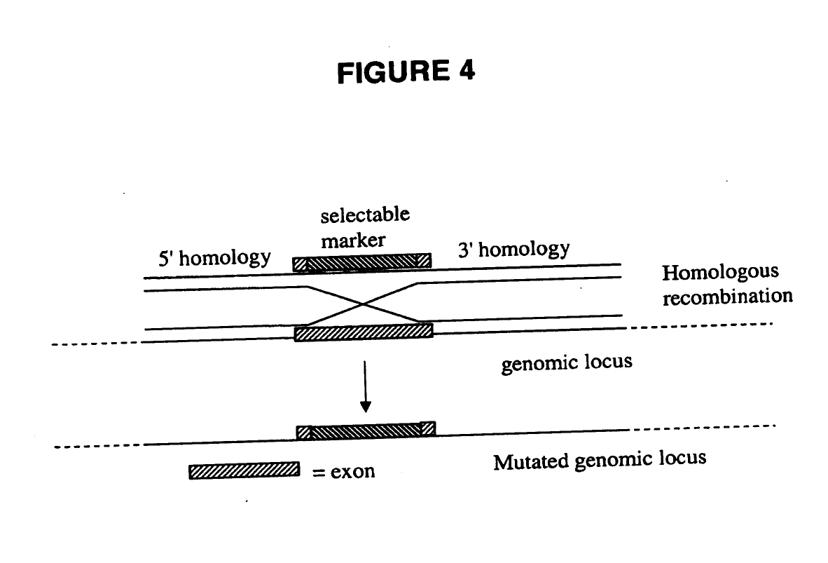

Gene targeting in mammalian cells is accomplished by homologous recombination between cloned genomic DNA and endogenous genomic sequence. The most commonly used gene targeting vector is the replacement vector in which a mammalian selectable marker is flanked by 5′ and 3′ genomic homology “arms” such that a functionally critical region of the target gene is either deleted or interrupted by the selectable marker. Upon transfection into mammalian cells (for example, mouse Embryonic Stem cells) homologous recombination may take place. The outcome is that endogenous genomic sequence is replaced by the targeting construct, leading to mutation of the target gene. For example, a single exon is interrupted by the selectable marker in the targeting construct and this mutation is incorporated into the genome following homologous recombination, as illustrated in FIG. 4.

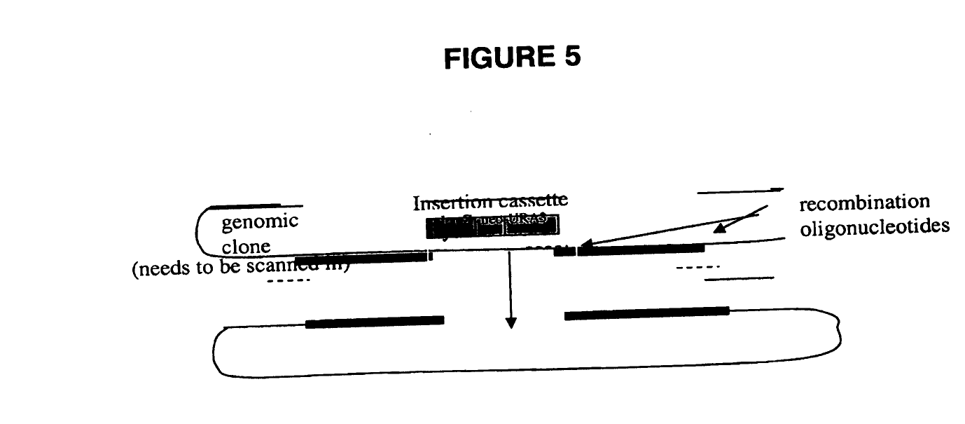

After obtaining and characterizing genomic clones from the target gene, the targeting vector is constructed. Homologous arms are subcloned into a standard plasmid vector and then the selectable marker, usually neo (which confers resistance to G418 in mammalian cells) is inserted to make the targeting vector. This subcloning is frequently complicated by a lack of useful restriction enzyme sites within the genomic clones. Also cloning may be complicated by the inclusion of functional units such as reporter genes within the selectable marker insert. Such time-consuming cloning steps can be greatly simplified by yeast using homologous recombination to construct the targeting vector. In the diagram below, recombination oligonucleotides are designed to overlap target sites between cloned genomic sequences, a yeast cloning vector and an insertion cassette. The insertion cassette contains a mammalian selectable marker (e.g. neo), a yeast selectable marker (e.g. URA3) and a reporter gene (e.g. LacZ). Yeast is transformed with the genomic clone, insertion cassette, yeast vector and recombination oligonucleotides. The yeast vector contains a yeast selectable marker (e.g. HIS3) which is different to the marker on the insertion cassette. Dual selection with URA3/HIS3 isolates yeast vector clones containing the insertion cassette and as this is dependant upon accurate recombination with the genomic homology arms. The final product is a completed targeting vector, as illustrated in FIG. 5.

The homologous recombination methodology for making the targeting vector is rapid and flexible, since the genomic arms and insertion cassette are positioned in a single step independant of restriction sites. Also the recombination method allows simultaneous modification of the insertion cassette with no additional subcloning. For example, the LacZ reporter gene in the illustratiojn above could easily be replaced with an in-frame cDNA insert to generate a “knock-in” where an exogenous cDNA (frequently the human homolog of a target mouse gene) is placed under the control of the target gene promoter.

The following examples further illustrate the present invention, but of course, should not be construed as in any way limiting its scope.

### EXAMPLE 1

### Use of Overlapping Oligonucleotides to Form Linkers

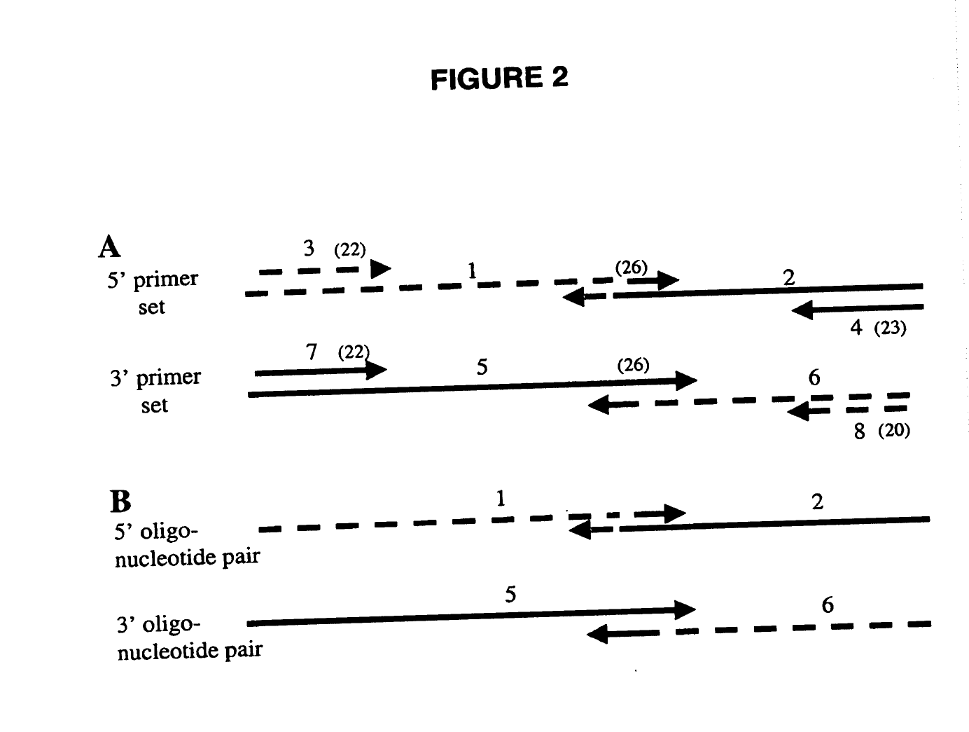

Strains, Growth Conditions, and Plasmids. The yeast strain used in this study was YEF473 MATa/α his3-Δ200/his3-Δ200 leu2-Δ1/leu2-Δ1 lys2-801/lys2-801 trp1-Δ63/trp1-Δ63 ura3-52/ura3-52 (Bi and Pringle, Mol. Cell. Biol. 16, 5264-5275, 1996). Yeast media have been described previously (Lillie and Pringle, J. Bacteriol. 143, 1384-1394, 1980; Guthrie and Fink, Methods Enzymol. 1, 1-933, 1991). The plasmids used were the high-copy pRS423 (Christianson et. al., Gene (Amst.) 110, 119-122, 1992), and pCDN (Aiyar et al, Mol. Cell Biochem. 131, 75-86, 1994), which were restricted with EcoRV (Promega) and NotI (Boerhinger Mannheim), respectively, extracted with phenol/chloroform, EtOH precipitated, and re-suspended in water. 0.1 μg of EcoRV-digested pRS423 (vector), 1.5 μg (a 1-μg equivalent of the DNA sequences to be cloned) of NotI-digested pCDN (insert), and either 1 μg of double-stranded linker (FIG. 2A and see below) or 1 μg of each of the indicated oligonucleotides (FIG. 2B), were transformed into YEF473 by the LiOAC procedure (Gyuris et al, Cell 75, 791-803, 1993). Transformants were selected on SC-His, which selects for strains that contain re-circularized plasmids. The transformants were pooled by adding approximately 1 ml of water to a plate of transfornants, and scraping the plate with a sterilized, disposable glass slide. The cell mixture was poured into an Eppendorf tube and microfuged for 30 seconds. Plasmid rescue was performed on the cell pellet as described (Hoffman and Winston, Gene 57, 267-272, 1987). The Escherichia coli strain was DH5α (GIBCO BRL) and standard media were used for plasmid preparation (Sambrook et al., Molecular Cloning: A Laboratory Manual. Cold Spring Harbor Laboratory Press, Cold Spring Harbor, N.Y., 1989). Plasmids were isolated from E. coli using the Qiaquick mini-prep procedure (Qiagen).

Oligonucleotides and PCR.

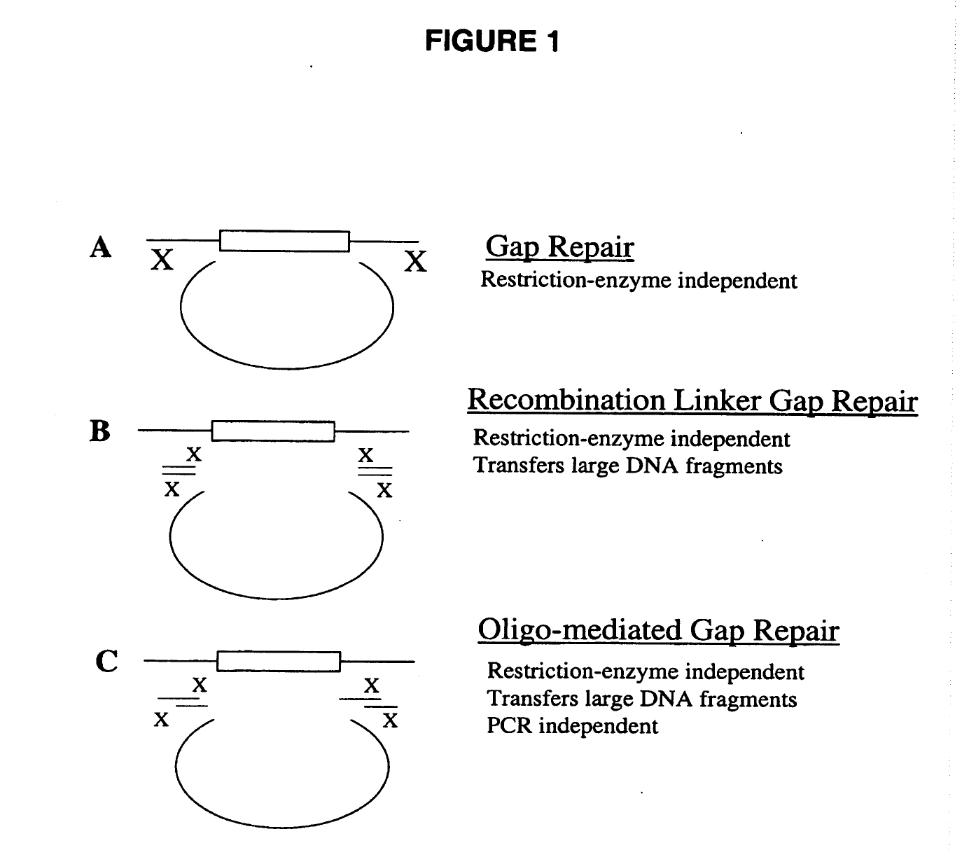

The oligonucleotides (Table 1) were synthesized on a Applied Biosystems 3948 Nucleic acids Synthesizer. Overlapping recombination linkers (FIG. 1) were synthesized using 0.1 μg of each annealing oligonucleotide and 2.5 U of Expand Polymerase (Boehringer Mannheim) in a 100 μl reaction for 20 cycles of: 94° C. for 1 minute, 60° C. for 30 seconds, 72° C. for 30 seconds. One μl of this reaction was used as substrate for the “extension” reaction using 0.1 μg of extending primers. This amplification was performed using the above conditions for 30 cycles. After amplification, 10 μl of each reaction was analyzed on a 2.5% agarose gel; the remainder of the extension reactions was extracted with phenol/chloroform, EtOH precipitated, and resuspended in water. Pre-annealing of the oligonucleotides was performed in 1× Expand buffer (Boehringer Mannheim) by heating to 90° C. for 1 minute, and slow-cooling the mixture to approximately 22° C. Sequence analysis was performed on an ABI 377 DNA Sequencer (Perkin Elmer).

Recombination with Double-Stranded Linkers, and Single-Stranded Oligonucleotides.

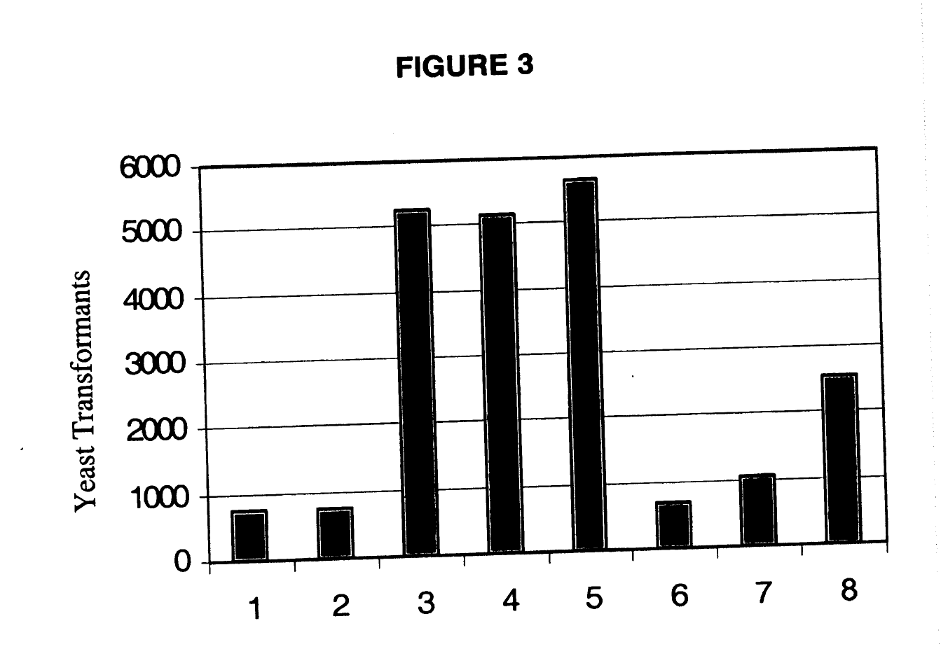

To determine if homologous recombination could be stimulated by oligonucleotides as well as double-stranded linkers, oligonucleotide sets were designed (FIG. 2A). Both pairs of the long oligonucleotides anneal through 26 complementary nucleotides at their 3′ ends. The regions of complementarity contain 12-14 nucleotides of pRS423 and pCDN sequences. When synthesized, the resulting double-stranded linkers have approximately 60 nucleotides of homology to both vector pRS423 and the NotI fragment to be transferred from pCDN. The reactions yielded approximately 2 μgs of product (FIG. 3). Additionally, 2 oligonucleotides (5′ SS and 3′ SS; Table 1) were designed that have 35 nucleotides of homology with both pRS423 and the NotI fragment of pCDN.

| | |
|---|---|
| Primer name (#)¹ | Primer sequence |

| | |
|---|---|
| 5′FA (1) | GAAATACCGCACAGATGCGTAAGGAGAAAATACC |
| | GCATCAGGAAATTGTAAACGTTAATATTGCGGCC |
| | GCTCTAG |
| 5′RA (2) | CCTCGGCCTCTGAGCTATTCCAGAAGTAGTGAGG |
| | AGGCTTTTTTGGAGGCCTAGAGCGGCCGCAATAT |
| | TAACGTTTAATACCGCACAGATGCGTAAGGCGGC |
| | CTCTGAGCTATTCCAGAAG |
| 5′FE (3) | CATTCTAGTTGTGGTTTGTCCAAACTCATCAATG |
| 5′RE (4) | TATCTTATCATGTCTGGATCGCGGCCGCCAGCTG |
| 3′FA (5) | CATTAAT |
| 3′RA (6) | GAAGAGCGCCCAATACGCAAACCGCCTCTCCCCG |
| | CGCGTTGGCCGATTCATTAATGCAGCTGGCGGCC |
| | GCGATCCTTCTAGTTGTGGTTTGTCCAACAGAGC |
| | GCCCAATACGCAAACC |
| 3′FE (7) | AAATACCGCATCAGGAAATTGTAAACGTTAATAT |
| 3′RE (8) | TGCGGCCGCTCTAGGCCTCCAAAAAAGCCTCCTC |
| 5′SS (9) | AC |
| 3′SS (10) | ATCAATGTATCTTATCATGTCTGGATCGCGGCCG |
| | CCAGCTGCATTAATGAATCGGCCAACGCGCGGGG |
| | AG |

¹These are SEQ ID Nos. 1-10, respectively.
Abbreviations: FA, forward annealing; RA, reverse annealing; FE, forward extending; RE, reverse extending; SS, single strand.
Underlining indicates nucleotides that anneal in a primer set (FIG. 1).
Bolding indicates nucleotides at the 5′ and 3′ ends of the NotI fragment being cloned from pCDN.

YEF473 was transformed with combinations of linearized pRS423 (vector), NotI-digested pCDN (insert), double-stranded linkers, and oligonucleotides. Cells transformed with vector, insert, and either double-stranded linkers (FIG. 3, column 3) yielded approximately 5-fold more transformants than did those transformed with vector alone or with vector and insert (FIG. 3 columns 1 and 2, respectively). Cells transformed with vector, insert, and complementary, substantially single-stranded oligonucleotides (FIG. 3, columns 4 and 8) yielded approximately 3-5 fold more transformants than did cells transformed with vector alone or vector plus insert. Annealing the oligonucleotide pairs did not significantly increase the number of transformants (FIG. 3, column 5). Cells transformed with only one oligonucleotide from each of the oligonucleotide pairs (FIG. 3, columns 6 and 7) yielded approximately the same number of transformants as did those tansformed with vector or vector plus insert. These results suggest that both double-stranded linkers and complementary, substantially, single-stranded oligonucleotide pairs can stimulate homologous recombination, whereas single-stranded DNAs do not.

Restriction and sequence analysis of recombinant clones. To examine the DNA products of the transformations, transformants were pooled and their plasmids rescued to E. coli. Restriction analysis was performed on 12 candidate plasmids from each of the transformations. Cells transformed with only vector and insert appeared to yield pRS423. The desired restriction patterns were obtained for: 11 of 12 plasmids from cells transformed with double-stranded linkers; 11 of 24 plasmids from cells transformed with combinations of complementary oligonucleotide pairs; 2 of 12 plasmids from cells transformed with pre-annealed oligonucleotides; and 0 of 24 plasmids from cells transformed with non-complementary oligonucleotides. Additionally, 67% ( 24/36) of the plasmids rescued from cells transformed with double-stranded linkers or complementary oligonucleotide appeared to be recombinant molecules, whereas only 3% ( 1/36) of the plasmids rescued from cells transformed with only vector and insert, or with non-complementary oligonucleotides appeared to be recombinant molecules.

To determine if double-stranded linkers or complementary oligonucleotide pairs yielded nucleotide-perfect recombination products, DNA sequence analysis was performed on several plasmids that gave the predicted restriction pattern. Sequencing primers were designed approximately 200 nucleotides upstream of the areas of recombination. 500-700 nucleotides of sequence were obtained per reaction. Analysis of three plasmids obtained with double-stranded linkers revealed that three of the recombination areas contained the correct sequence, and three did not. The errors were: a single-base substitution, three single-base substitutions, and a deletion of undetermined size. All of the errors occurred in the linker sequences. Thus, 50% of the recombination junctions contained the desired sequence, resulting in 1 correct plasmid. Analysis of 5 plasmids obtained from complementary oligonucleotides revealed that 8 of 10 recombination areas contained the correct sequence, and 2 did not. The sequence errors were a single-base substitution, and a 4-base deletion. Both errors were in the region of the oligonucleotides, and were different from those created by the double-stranded linkers. Thus, 80% of the recombination junctions contained the desired sequence, resulting in 3 correct plasmids.

The experiment as described in FIG. 3 column 4 using pRS423, pCDN, and oligos 1,2,5, and 6 was performed successfully 3 times. The experiment as described in FIG. 3, column 5 using pRS423, pCDN, and oligos 1,2,5, and 6 was performed successfully 2 times. The experiment as described in FIG. 3 column 8 using pRS423, pCDN. and oligos 2, 6, 9, and 10 was performed successfully 2 times. One experiment, using similarly designed oligonucleotides but different vector and insert sequences, and performed only once, failed.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

While this invention has been described with an emphasis upon preferred embodiments, it will be obvious to those of ordinary skill in the art that variations in the preferred devices and methods may be used and that it is intended that the invention may be practiced otherwise than as specifically described herein. Accordingly, this invention includes all modifications encompassed within the spirit and scope of the invention as defined by the claims that follow.

What is claimed:

1. A method of inserting an insert polynucleotide into a target nucleic acid having a first end and a second end by homologous recombination comprising:
inserting into a recombination-competent cell the following first nucleic acids:
one or more insert polynucleotides each comprising an insert segment, and
at least one linker comprising (i) a substantially single-stranded recombination sequence, (ii) a recombination sequence of no more than 25 base pairs, or (iii) a combined length of no more than 45 nucleotides; and
generating within the cell a nucleic acid containing the insert segments inserted between the first and second ends in an order defined by recombination sequences found in the target nucleic acid and the first nucleic acids.

2. The method of claim 1, comprising inserting an insert polynucleotide into genomic nucleic acid.

3. A method of inserting an insert polynucleotide into of claim 1, the method comprising:
inserting into a recombination-competent cell the following first nucleic acids:
the one or more insert polynucleotides each comprising an insert segment,
a target nucleic acid comprising a vector-related nucleic acid having the first end and the second end,
the at least one linker comprising (i) a substantially single-stranded recombination sequence, (ii) a recombination sequence of no more than 25 base pairs, or (iii) a combined length of no more than 45 nucleotides; and
generating within the cell a vector containing the insert segments inserted between the first and second ends in an order defined by recombination sequences found in the first nucleic acids.

4. The method of claim 1, comprising inserting a single insert polynucleotide-derived insert segment into the vector.

5. The method of claim 1, comprising inserting multiple polynucleotide-derived insert segments in a defined orientation dictated by recombination sequences.

6. The method of claim 1, comprising using at least two linkers each comprising a substantially single-stranded recombination sequence.

7. The method of claim 1, wherein at least one of the linkers consists of a single nucleic acid.

8. The method of claim 1, wherein at least one of the linkers consists of two substantially single-stranded nucleic acids with a complementary overlap between the two.

9. The method of claim 1, wherein at least one end of the insert segment is in the interior of the insert polynucleotide.

10. The method of claim 1, wherein the at least one linker comprises a substantially single-stranded recombination sequence.

11. The method of claim 1, wherein the at least one linker comprises a recombination sequence of no more than 25 base pairs.

12. The method of claim 1, wherein the at least one linker comprises a recombination sequence of no more than 20 base pairs.

13. The method of claim 1, wherein the at least one linker comprises a recombination sequence of no more than 15 base pairs.

14. The method of claim 1, wherein the at least one linker comprises a combined length of no more than 45 nucleotides.

15. The method of claim 1, wherein the at least one linker comprises a combined length of no more than 40 nucleotides.

16. The method of claim 1, wherein the vector generated is animal gene replacement targeting vector comprising an animal-specific selectable marker and a selectable marker specific for the recombination-competent cell.

17. A method of cloning or subcloning an insert nucleic acid comprising:
isolating an insert polynucleotide comprising the (i) insert nucleic acid and, (ii) at least at one end of the insert nucleic acid, an additional polynucleotide portion;
inserting into a recombination-competent cell the following first nucleic acids:
the insert polynucleotide,
a vector-related nucleic acid having a first end and a second end,
at least one linker comprising (i) a substantially single-stranded recombination sequence, (ii) a recombination sequence of no more than 25 base pairs, or (iii) a combined length of no more than 45 nucleotides; and
generating within the cell a vector containing the insert nucleic acid inserted between the first and second ends in an order defined by recombination sequences found in the first nucleic acids.

18. The method of claim 17, wherein the first linker nucleic acids are synthesized without the use of an enzyme-catalyzed amplification reaction.

19. A method of cloning or subcloning an insert nucleic acid:
isolating (i) a first insert polynucleotide comprising (a) a first insert segment that defines a portion of the insert nucleic acid and, at one or both ends of the first insert segment, (b) additional polynucleotide sequence, and (ii) one or more additional insert polynucleotides comprising additional insert segments that define portions of the insert nucleic acid, wherein the insert segments can be aligned to generate the insert nucleic acid;
inserting into a recombination-competent cell the following first nucleic acids:
the first insert polynucleotides and the additional insert polynucleotides,
a vector-related nucleic acid having a first end and a second end,
at least one linker comprising (i) a substantially single-stranded recombination sequence, (ii) a recombination sequence of no more than 25 base pairs, or (iii) a combined length of no more than 45 nucleotides; and
generating within the cell a vector containing the insert nucleic acid inserted between the first and second ends in an order defined by recombination sequences found in the first nucleic acids.


SEQUENCE LISTING (10 sequences)

SEQ ID NO: 1 - DNA, 75 bp, Artificial Sequence
  Wherein in the nucleotide sequence represents a forward annealing sequence
gaaataccgcacagatgcgtaaggagaaaataccgcatcaggaaattgtaaacgttaatattgcggccgctctag

SEQ ID NO: 2 - DNA, 76 bp, Artificial Sequence
  Wherein in the nucleotide sequence represents a reverse annealing sequence
cctcggcctctgagctattccagaagtagtgaggaggcttttttggaggcctagagcggccgcaatattaacgttt

SEQ ID NO: 3 - DNA, 22 bp, Artificial Sequence
  Wherein in the nucleotide sequence represents a forward extending sequence
aataccgcacagatgcgtaagg

SEQ ID NO: 4 - DNA, 23 bp, Artificial Sequence
  Wherein in the nucleotide sequence represents a reverse extending sequence
cggcctctgagctattccagaag

SEQ ID NO: 5 - DNA, 75 bp, Artificial Sequence
  Wherein in the nucleotide sequence represents a forward annealing sequence
cattctagttgtggtttgtccaaactcatcaatgtatcttatcatgtctggatcgcggccgccagctgcattaat

SEQ ID NO: 6 - DNA, 75 bp, Artificial Sequence
  Wherein in the nucleotide sequence represents a reverse annealing sequence
gaagagcgcccaatacgcaaaccgcctctccccgcgcgttggccgattcattaatgcagctggcggccgcgatcc

SEQ ID NO: 7 - DNA, 22 bp, Artificial Sequence
  Wherein in the nucleotide sequence represents a forward extending sequence
ttctagttgtggtttgtccaac

SEQ ID NO: 8 - DNA, 21 bp, Artificial Sequence
  Wherein in the nucleotide sequence represents a reverse extending sequence
agagcgcccaatacgcaaacc

SEQ ID NO: 9 - DNA, 70 bp, Artificial Sequence
  Wherein in the nucleotide sequence represents a single strand sequence
aaataccgcatcaggaaattgtaaacgttaatattgcggccgctctaggcctccaaaaaagcctcctcac

SEQ ID NO: 10 - DNA, 70 bp, Artificial Sequence
  Wherein in the nucleotide sequence represents a single strand sequence
atcaatgtatcttatcatgtctggatcgcggccgccagctgcattaatgaatcggccaacgcgcggggag